(19) United States
(12) Patent Application Publication
(10) Pub. No.: US 2014/0166004 A1
(43) Pub. Date: Jun. 19, 2014

(54) NEBULIZER WITH INTEGRATED BREATHING INCENTIVE

(71) Applicant: CAREFUSION 303, INC., San Diego, CA (US)

(72) Inventors: Brian PIERRO, Yorba Linda, CA (US); Christopher VARGA, Laguna Hills, CA (US); Thomas DILLINGHAM, Aliso Viejo, CA (US); Khalid MANSOUR, Corona, CA (US)

(73) Assignee: CareFusion 303, Inc., San Diego, CA (US)

(21) Appl. No.: 13/720,870

(22) Filed: Dec. 19, 2012

(51) Int. Cl. A61M 16/14; A61M 16/00

(52) U.S. Cl. CPC A61M 16/14; A61M 16/0057
USPC 128/203.12

(57) ABSTRACT

A nebulizer comprises a body, a patient interface, an air inlet, an air outlet, and at least one breathing incentive. The air inlet is coupled with the body and configured for admittance of ambient air into the body in response to inhalation through the patient interface. The air outlet is coupled with the body and configured for discharging exhaled breath. The at least one breathing incentive is integrated with the nebulizer and configured to provide feedback in response to the inhalation. The feedback describes a propriety of a flow rate of the inhalation so as to assist a patient using the nebulizer in achieving a therapeutically effective flow rate for delivery of a medical aerosol.

### BACKGROUND

Nebulizers can be used for treating living beings that are capable of spontaneous breathing or living beings that are using controlled ventilation mechanisms, among other things. Nebulizers can be used to create a fine spray of medication with small particles of medication suspended in gas (also referred to herein as “medical aerosol”) that can be inhaled by the living being. Medication in the form of liquid, among other things, can be placed inside of the nebulizer. The nebulizer can be used to mix gas with the medication inside of the nebulizer to create the medical aerosol that is delivered to the living being through a mouth piece, mask, face-tent or the like associated with a patient interface of the nebulizer.

### BRIEF DESCRIPTION OF DRAWINGS

The accompanying drawings, which are incorporated in and form a part of this specification, illustrate various embodiments and, together with the Description of Embodiments, serve to explain principles discussed below. The drawings referred to in this brief description of the drawings should not be understood as being drawn to scale unless specifically noted.

FIG. 1A depicts a block diagram of a nebulizer with at least one integrated breathing incentive along with a depiction of an inhalation path, according to some embodiments.

FIG. 1B depicts a block diagram of the nebulizer of FIG. 1A along with a depiction of an exhalation path, according to some embodiments.

FIG. 2 depicts an integrated breathing incentive which utilizes a float to provide breathing incentive feedback, according to some embodiments.

FIG. 3 depicts an integrated breathing incentive which utilizes a whistle to provide breathing incentive feedback, according to some embodiments.

FIG. 4 depicts an integrated breathing incentive which utilizes a reed to provide breathing incentive feedback, according to some embodiments.

FIG. 5 depicts an integrated breathing incentive which utilizes a rotating wheel to provide breathing incentive feedback, according to some embodiments.

FIGS. 6A and 6B depict an integrated breathing incentive which utilizes a color changing material to provide breathing incentive feedback, according to some embodiments.

FIGS. 7A and 7B illustrate a flow diagram for an example method of administering medical aerosol, according to various embodiments.

### DESCRIPTION OF EMBODIMENTS

Reference will now be made in detail to various embodiments, examples of which are illustrated in the accompanying drawings. While various embodiments are discussed herein, it will be understood that they are not intended to be limiting. On the contrary, the presented embodiments are intended to cover alternatives, modifications and equivalents, which may be included within the spirit and scope the various embodiments as defined by the appended claims. Furthermore, in this Description of Embodiments, numerous specific details are set forth in order to provide a thorough understanding. However, embodiments may be practiced without one or more of these specific details. In other instances, well known methods, procedures, and components have not been described in detail as not to unnecessarily obscure aspects of the described embodiments.

### Overview of Discussion

Nebulizers can be used for creating medical aerosol for treating living beings. Discussion begins with the description of a block diagram of a nebulizer which includes one or more integrated breathing incentives. Example inhalation and exhalation paths are described. A variety of breathing incentives are then described. The nebulizer and breathing incentives are then further described in conjunction with an example method of administering medical aerosol.

### Terms

The term “patient” describes a living being, typically human, to whom a medication is provided via a nebulizer.

The term “nebulizer” describes a device that creates medical aerosol (nebulized medicine which may be mixed with ambient air) which can be inhaled in response to a patient inhaling through a mouthpiece associated with a patient interface of the nebulizer. A nebulizer may constantly produce medical aerosol, in some embodiments. In other embodiments, a nebulizer may produce/increase the production of the medical aerosol in response to inhalation of the patient who is using the nebulizer while ceasing/reducing production of the medical aerosol in response to exhalation of the patient or in response to a cessation of the inhalation. A variety of methods and devices are known in the art for nebulizing medication, therefore discussion herein will not focus details of the process of nebulization, as such focus would tend to obscure discussion of other features described herein which may be integrated with a nebulizer in order to provide breathing incentive feedback.

The term “medical amount” is defined as an amount of medical aerosol that would be used for treating a patient using that type of medical aerosol.

The term “therapeutically effective flow rate” is defined as a particular predetermined flow rate or range of flow rates (with upper and lower bounds) of inhalation which a patient is required to achieve in order to assure delivery of the medical amount of medical aerosol into the lungs of the patient. The therapeutically effective flow rate may vary between medications used and/or patients, but in general tends to fall between 5 liters/minute and 50 liters per minute as lower and upper bounds of what is considered therapeutic, and in many cases is at or near 15 liters/minute. Flow rates that are lower than the therapeutically effective flow rate do not deliver enough medical aerosol to a patient and/or do not deliver the medical aerosol deep enough into the lungs of a patient, while higher flow rates may waste medication or result in deposition that is not efficient (e.g., medical aerosol may be deposited in undesirable areas like the mouth or alveoli).

The term “breathing incentive” is defined as a non-electrical mechanism that interacts with flow (which may include flow of admitted ambient air, flow of medical aerosol, and/or flow of exhaled breath) through a nebulizer and provides analog breathing incentive feedback regarding an inhalation or exhalation flow rate of the patient. Breathing incentive feedback is provided across a range of flow rates, and is thus more than just an indication of whether a threshold is met or not met. For example, a threshold indicator would only indicate whether or not a threshold, such as desired inhalation flow rate had been met or not met. However, breathing incentives as described herein typically provide feedback across a wide spectrum of flow rates (e.g., between 0 and 100 liters/minute, in some embodiments or some therapeutically effective coached range such as between 10 and 50 liters/minute in some embodiments). The wide range may in some embodiments encompass and extend below and above the lower and upper bounds of what is considered therapeutically effective, and thus provide feedback about meeting a threshold as well as feedback about how far above or below a threshold a flow rate is and/or how close to or far from an target flow rate a patient is. In some cases there may be a target flow rate (e.g., 30 liters per minute in one embodiment) along with upper and lower thresholds that demarcate a therapeutically effective range, and the breathing incentives described herein provide coaching toward that target flow rate even when the flow rate is within the bounds of the upper and lower thresholds. In at least these manners, such breathing incentive feedback provides feedback regarding the propriety of flow with respect to a desired target (e.g., the therapeutically effective flow rate). It is appreciated that such breathing incentive feedback may be associated with an inhalation flow rate, an exhalation flow rate, or both.

### Example Nebulizer with Integrated Breathing Incentive

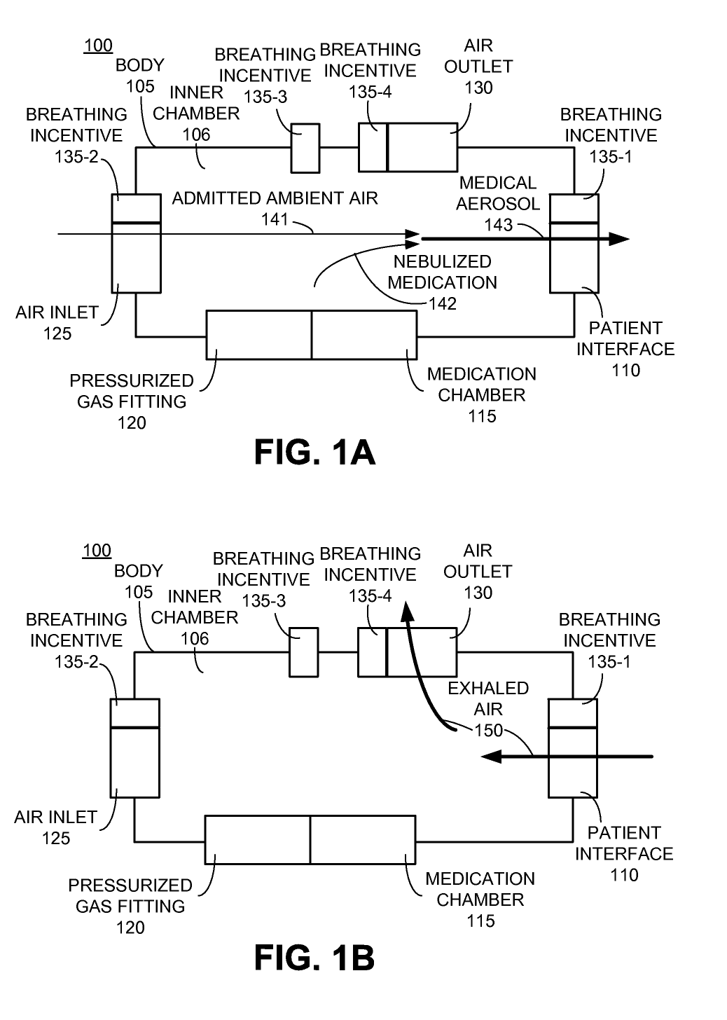

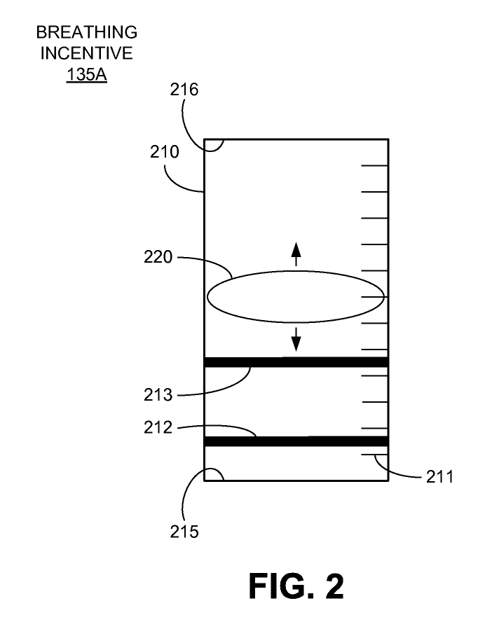

FIG. 1A depicts a block diagram of an example nebulizer 100 with at least one integrated breathing incentive 135 along with a depiction of an inhalation path, according to some embodiment. As depicted in FIG. 1, nebulizer 100 includes a body 105, a patient interface 110, a medication chamber 115, a pressurized gas fitting 120, an air inlet 125, an air outlet 130, and at least one integrated breathing incentive 135.

Body 105 is typically composed of metal, plastic, or some combination thereof. Body 105 forms both an outer shell and an inner chamber 106 in which admitted ambient air 141 and nebulized medication 142 mix into a medical aerosol 143 prior to being inhaled by a patient through patient interface 110 (it should be appreciated that a low rate of inhalation may result in a medical aerosol 143 which contains no admitted ambient air 141 or very little admitted ambient air 141).

A patient interface 110 is coupled with the body and provides an opening through which medical aerosol 143 may be inhaled into the lungs of a patient. It is appreciated that, in various embodiments, patient interface 110 may be used as a mouthpiece or as a coupling point to which a mouthpiece and/or a tube and mouthpiece (neither depicted) may be coupled.

Medication chamber 115 is coupled with body 105 and configured to hold a medication to be nebulized. The medication is typically in liquid form, but may be in other forms.

Pressurized gas fitting 120 is coupled with body 105 and configured to receive pressurized gas with which to nebulize the medication into a nebulized medication. Many techniques for nebulizing a medication with a pressurized gas are known and practiced. For example, in some embodiments, the received pressurized gas may be directed from pressurized gas fitting 120 such that it shears across a surface of a medication disposed in medication chamber 115 and then enters the inner chamber 106 as nebulized medication 142.

Air inlet 125 is coupled with the body 105 and permits admittance of ambient air into the body in response to inhalation through the patient interface. The admitted ambient air 141 admitted through air inlet 125 may be admitted through one or more openings that form air inlet 125. In some embodiments, such openings include one-way valves which permit ambient air to be admitted into chamber 106, but do not permit admitted ambient air 141, nebulized medication 142, or medical aerosol 143 to flow outward from inner chamber 106. In other embodiments, such openings are non-valved. Although only one air inlet 125 is depicted in FIGS. 1A and 1B, in some embodiments, more than one air inlet 125 may be coupled with body 105.

FIG. 1B depicts a block diagram of the example nebulizer 100 of FIG. 1A along with a depiction of an exhalation path, according to some embodiment. Air outlet 130, when included, is coupled with body 105 and configured for discharging exhaled air 150 that is received via exhalation of a patient into patient interface 110. In some embodiments, air outlet 130 comprises a one-way valve which opens in response to directional flow of exhaled air 150 but does not open to permit admittance of ambient air into inner chamber 106. For purposes of clarity of illustration, air outlet 130 is depicted as being separate from other components of nebulizer 100, but it should be appreciated that air outlet 130 may be incorporated within patient interface 110, air inlet 125, and/or as a portion of one or more components associated with nebulizer 100 (such as a portion of a mouth piece or tubing coupled with patient interface 110).

With continued reference to FIGS. 1A and 1B, at least one breathing incentive 135 is integrated with nebulizer 100. In FIGS. 1A and 1B three integrated breathing incentives 135-1, 135-2, 135-3, and 135-4 are depicted. However, it should be appreciated that in some embodiments more integrated breathing incentives 135 may be included or as few as one integrated breathing incentive 135 may be included. By “integrated,” what is meant is that the breathing incentive 135 is manufactured or assembled to be a portion of nebulizer 100 such that the entirety of nebulizer 100 including body 105 and the integrated breathing incentive(s) 135 may be easily held in the hand of a patient during use of nebulizer 100. In various embodiments, the included integrated breathing incentive(s) may be coupled with air inlet 125 (e.g., 135-1), with patient interface 110 (e.g., 135-2), with body 105 (e.g., 135-3), and/or with air outlet 130 (e.g., 135-4).

In one embodiment, an included integrated breathing incentive 135 is configured to provide breathing incentive feedback in response to inhalation through patient interface 110. Such breathing incentive feedback provided during inhalation of a patient describes a propriety of a flow rate of the inhalation so as to assist a patient using the nebulizer in achieving a therapeutically effective flow rate for delivery of a medical aerosol. By propriety, what is meant is that the feedback provides indication of where the air flow is on a spectrum which includes a target therapeutically effective flow rate (which may be a flow rate range) as well as flow rates both above and below the therapeutically effective flow rate. Such feedback can assist a caregiver in coaching the patient, or can assist in self-coaching the patient, to achieve inhalation at the therapeutically effective flow rate.

In one embodiment, one or more of the included integrated breathing incentive(s) is configured to provide breathing incentive feedback in response to exhalation through patient interface 110. Such feedback can assist a caregiver and/or patient in evaluating whether or not the patient is exhaling properly thorough nebulizer 100 and/or at a desired flow rate of exhalation.

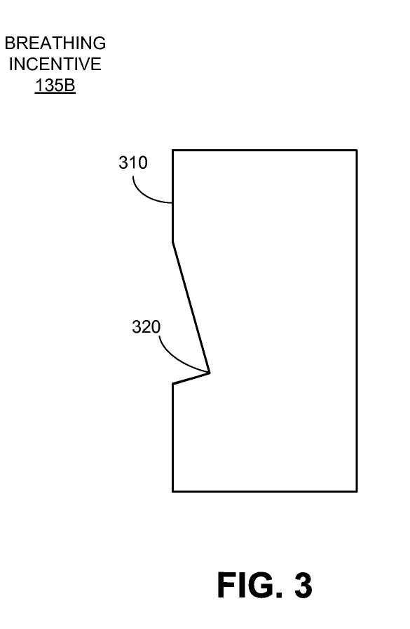

FIGS. 2, 3, 4, 5, 6A, and 6B illustrate a variety of integrated breathing incentives which may be used as an integrated breathing incentive 135, in various embodiments. It should be appreciated that these integrated breathing incentives may be used alone or in various combinations with one another, in various embodiments of nebulizer 100.

FIG. 2 depicts an integrated breathing incentive 135A which utilizes a float 220 to provide a visible breathing incentive feedback, according to some embodiments. In one embodiment, breathing incentive 135A functions as a volumetric spirometer, through which a portion of flow through nebulizer 100 is diverted, and in which float 220 moves up and down across a variety of positions between bottom 215 and top 216 of housing 210 in response to variations in an inhalation flow rate through patient interface 110 of nebulizer 100. In some embodiments all or a portion of housing 210 may be made of a transparent material. Float 220 may take the shape of a ball, an ovoid, a disk, or some other shape. Visible graduations 211 describe units of air flow volume (such as tens of liters per minute), and float 220 moves with respect to visible graduations 211 to provide visible feedback to a patient and/or caregiver regarding flow rate associated with the patient's inhalation through patient interface 110. Additional visible graduations 212 and 213 may be provided which show a lower bound 212 and an upper bound 213 that are indicative of a range of positions of movable float 220 associated with a therapeutically effective flow rate for delivery of a medical aerosol. Visible graduations 212 and 213 provide more prominent visible feedback by indicating that a patient is in/maintaining a therapeutically effective flow rate when float 220 is between visible graduations 212 and 213. In one embodiment, the locations of visible graduations 212 and 213 are fixed, while in another embodiment the locations of visible graduations 212 and 213 may be adjusted. In some embodiments, integrated breathing incentive 135A, may be similarly implemented to provide visible feedback with respect to the exhalation flow rate of a patient.

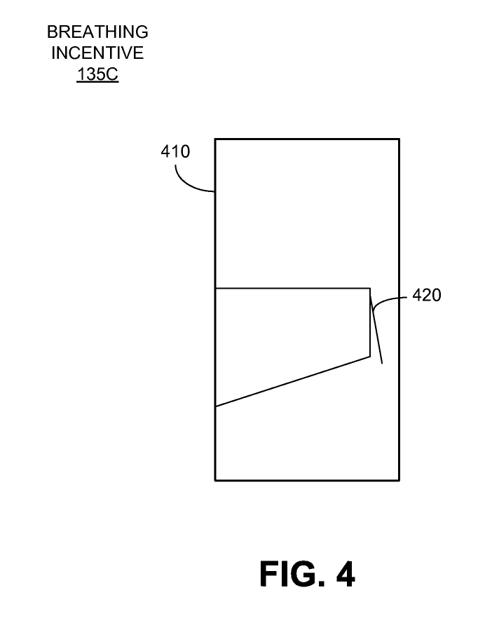

FIG. 3 depicts an integrated breathing incentive 135B which utilizes a whistle 320 to provide breathing incentive feedback, according to some embodiments. Whistle 320 is, in one embodiment, configured as a notch within a body 310 through which a portion of flow through nebulizer 100 is diverted. Whistle 320 generates a whistling sound via fluid mechanical motion of the diverted flow through whistle 320. It is appreciated that other forms of whistles may be implemented besides the whistle 320 depicted in FIG. 3. Whistle 320 acts as an audible signal generator and generates an audible signal in the form of a whistling sound which, in some embodiments, varies in response to variations in the inhalation flow rate through patient interface 110 of nebulizer 100. For example, in some embodiments, whistle 320 is designed such that a slower flow rate will result in a lower frequency whistle while a higher flow rate will result in a higher frequency whistle. In other embodiments, whistle 320 is designed such that a whistling sound begins at the lower end of a range associated with a therapeutically effective flow rate and ceases at the upper end of a range associated with a therapeutically effective flow rate. A caregiver or patient may be trained to identify a frequency or range of frequencies of whistling generated by whistle 320 which is/are associated with a therapeutically effective flow rate for delivery of medical aerosol via nebulizer 100. By listening to the frequency of whistling generated by whistle 320, a caregiver or patient may identify whether or not an inhalation flow rate is adequate, needs to increase, or needs to decrease, without looking at nebulizer 100. This can be advantageous in a dark room, for a caregiver or patient who has poor/no vision, or if the caregiver is busy performing another task while a patient is using nebulizer 100. In some embodiments, integrated breathing incentive 135B, may be similarly implemented to provide audible feedback with respect to the exhalation flow rate of a patient.

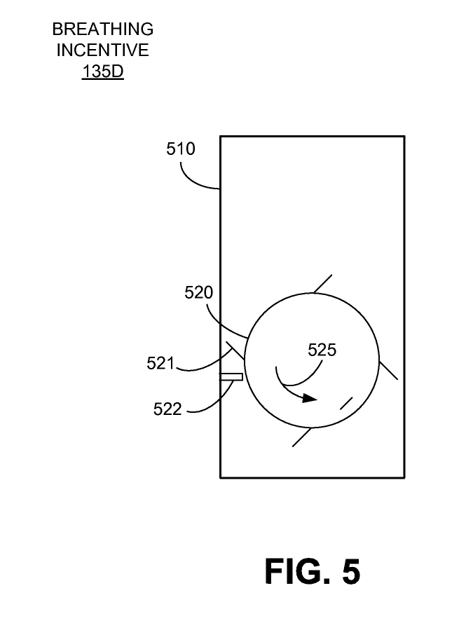

FIG. 4 depicts an integrated breathing incentive 135C which utilizes a reed 420 to provide breathing incentive feedback, according to some embodiments. Reed 420 is, in one embodiment, configured within a body 410 through which a portion of flow through nebulizer 100 is diverted. Reed 420 generates a vibratory sound or tone via fluid mechanical motion of the diverted flow across reed 420. Reed 420 acts as an audible signal generator and generates an audible signal in the form of a vibratory sound which, in some embodiments, varies in response to variations in the inhalation flow rate through patient interface 110 of nebulizer 100. For example, in some embodiments, reed 420 is designed such that a slower flow rate will result in a lower frequency vibratory sound while a higher flow rate will result in a higher frequency vibratory sound. In other embodiments, reed 420 is designed such that a vibratory sound begins at the lower end of a range associated with a therapeutically effective flow rate and ceases at the upper end of a range associated with a therapeutically effective flow rate. A caregiver or patient may be trained to identify a frequency or range of frequencies of vibratory sound generated by reed 420 which is/are associated with a therapeutically effective flow rate for delivery of medical aerosol via nebulizer 100. By listening to the frequency of vibratory sound generated by reed 420, a caregiver or patient may identify whether or not an inhalation flow rate is adequate, needs to increase, or needs to decrease, without looking at nebulizer 100. This can be advantageous in a dark room, for a caregiver or patient who has poor/no vision, or if the caregiver is busy performing another task while a patient is using nebulizer 100. In some embodiments, integrated breathing incentive 135C, may be similarly implemented to provide audible feedback with respect to the exhalation flow rate of a patient.

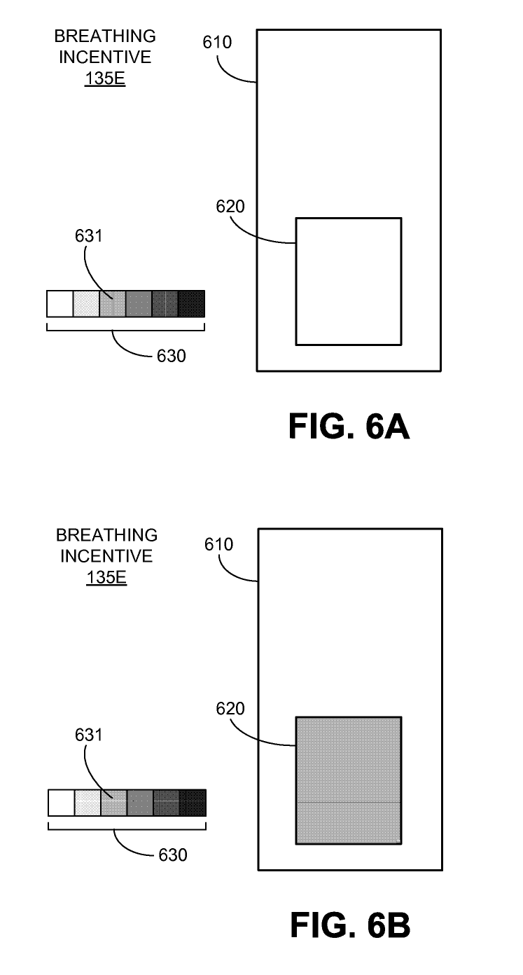

FIG. 5 depicts an integrated breathing incentive 135D which utilizes a rotatable wheel 520 to provide breathing incentive feedback, according to some embodiments. Wheel 520 is, in one embodiment, configured within a body 510 through which a portion of flow through nebulizer 100 is diverted. Wheel 520 generates a clicking sound via fluid mechanical motion of the diverted flow which interacts with fins 521 and to induce wheel 520 to rotate in the direction shown by arrow 525. As fins 510 contact post 522 a clicking sound is generated. Wheel 520 acts as an audible signal generator and generates an audible signal in the form of a clicking sound which, in some embodiments, varies in response to variations in the inhalation flow rate through patient interface 110 of nebulizer 100. For example, in some embodiments, wheel 520 is designed such that a slower flow rate will result in a lower frequency clicking sound while a higher flow rate will result in a higher frequency clicking sound. In other embodiments, wheel 520 is designed such that rotation, and thus the clicking sound, begins at the lower end of a range associated with a therapeutically effective flow rate range associated with a therapeutically effective flow rate. A caregiver or patient may be trained to identify a frequency or range of frequencies of clicking sound generated by wheel 520 which is/are associated with a therapeutically effective flow rate for delivery of medical aerosol via nebulizer 100. By listening to the frequency of clicking sound generated by wheel 520, a caregiver or patient may identify whether or not an inhalation flow rate is adequate, needs to increase, or needs to decrease, without looking at nebulizer 100. This can be advantageous in a dark room, for a caregiver or patient who has poor/no vision, or if the caregiver is busy performing another task while a patient is using nebulizer 100. In some embodiments, integrated breathing incentive 135D, may be similarly implemented to provide audible feedback with respect to the exhalation flow rate of a patient.

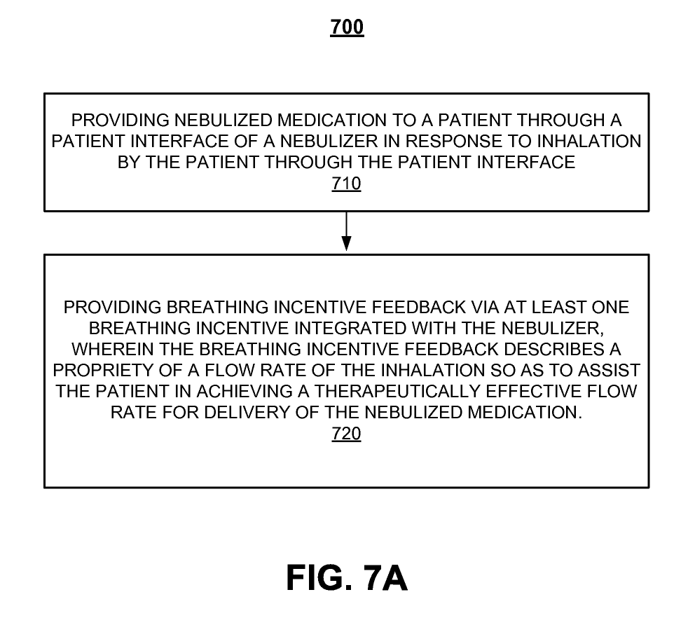

FIGS. 6A and 6B depict an integrated breathing incentive 135E which utilizes a color changing material 620 to provide a visible breathing incentive feedback, according to some embodiments. Color changing material 620 is, in one embodiment, disposed as part of, within, or upon a body 610 through which a portion of flow through nebulizer 100 is diverted or else normally flows. In some embodiments, a component of nebulizer 100, such as, but not limited to, body 105, patient interface 110, air inlet 125, or air outlet 130 may include or may be constructed all or in part from color change material 620. Spectrum of colors 630 represents a spectrum of possible colors of color change material 620, according to one embodiment. While color 631 represents a color that is associated with a therapeutically effective flow rate, according to one embodiment. In some embodiments, color changing material 620 is integrated with nebulizer 100 and configured to generate a visible color change which varies in response to variations in inhalation flow rate, exhalation flow rate, or some combination thereof.

In some embodiments, color change material 620 changes color in response to changes in temperature. In operation of nebulizer 100 medical aerosol 143 is cooler in temperature than exhaled air 150. In one embodiment, integrated breathing incentive 135E is positioned, such as at the location represented by integrated breathing incentive 135-1, such that a temperature sensitive color change material 620 represents a color that is an aggregation of these cool and warm temperatures and is designed such that color 631 represents a balance of inhalation and exhalation flow rates which is designed to represent a therapeutically effective flow rate for delivery of medical aerosol via nebulizer 100.

In some embodiments, color change material 620 changes color in response to changes in concentration of a chemical presence. For example, changes in color of color change material 620 may occur in response to changes in the concentration of carbon dioxide present in a flow across/through color change material 620. In operation of nebulizer 100, medical aerosol 143 is lower in carbon dioxide than exhaled air 150. In one embodiment, a chemically sensitive color change material 620 may be positioned and designed such that the color change material 620 represents a color that is an aggregation of these higher and lower presences of carbon dioxide (or some other chemical) and is further designed such that color 631 represents a balance of inhalation and exhalation flow rates which is deemed represent a therapeutically effective flow rate for delivery of medical aerosol via nebulizer 100.

A caregiver or patient may be trained to identify a color 631 or range of colors in color spectrum 630 which is/are associated with a therapeutically effective flow rate for delivery of medical aerosol via nebulizer 100. By viewing a color of color change material 620, a caregiver or patient may identify whether or not an inhalation flow rate and/or exhalation flow rate is adequate, needs to increase, or needs to decrease. As one non-limiting example, the color of color change material 620 may lighten (e.g., become whiter or more transparent) when an inhalation flow rate is lower and may darken when the inhalation flow rate is higher. As another non limiting example, a low flow rate may be indicated by color change material 620 by a color such as yellow, while a therapeutically effective flow rate is indicated by a color such as green, and a flow rate which is too high may be indicated by a color such as blue. It is appreciated that a variety of colors and meanings may be assigned, depending on the type of color change material used.

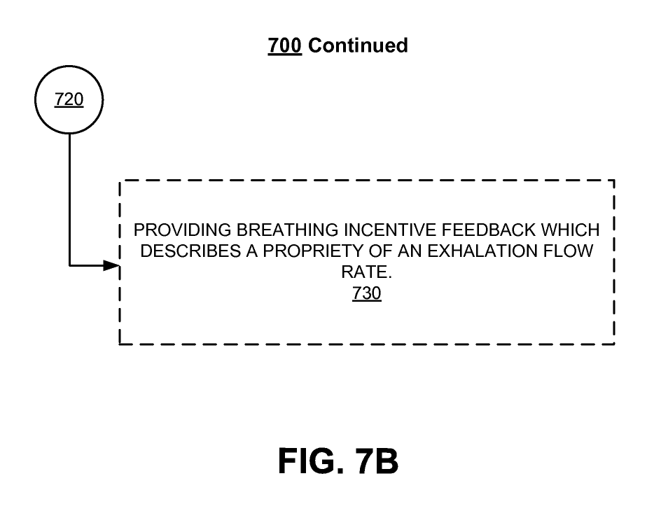

FIGS. 7A and 7B illustrate a flow diagram 700 for a method of administering medical aerosol according to one embodiment.

At 710 of flow diagram 700, in one embodiment, a medical aerosol 143 is provided to a patient through a patient interface 110 of a nebulizer 100 in response to inhalation by a patient through the patient interface 100.

At 720 of flow diagram 700, in one embodiment, a breathing incentive feedback is provided via at least one breathing incentive 135 integrated with the nebulizer 100. The breathing incentive feedback may be an audible feedback, visible feedback, or some combination thereof. In one embodiment, the breathing incentive feedback describes a propriety of a flow rate of the inhalation so as to assist the patient in achieving a therapeutically effective flow rate for delivery of the medical aerosol.

With reference to FIGS. 2 and 6A, in some embodiments, providing breathing incentive feedback comprises providing visible feedback via an integrated breathing incentive 135, in response to the inhalation through patient interface 110. Visible feedback may be provided by diverted airflow moving a movable float within a graduated housing that is integrated some portion of nebulizer 100. The float is movable to a variety of positions in the housing in response to variations in the inhalation flow rate. Visible feedback may also be provided by the changing of a color of a color change material. For example, visible feedback may be provided by generating a visible color change with a color changing material integrated with nebulizer 100 and configured to generate a visible color change which varies in response to variations in the inhalation flow rate, the exhalation flow rate, or some combination thereof. Such visible feedback, as illustrated in one or both of FIGS. 2 and 6A, may be used in isolation or in combination and/or in conjunction with other visible feedback. For example, a movable float may be used in conjunction with a color change material. In some embodiments, audible breathing incentive feedback may be provided in addition to one or more means of visible feedback. In some embodiments, the audible breathing incentive feedback varies with respect to and in response to variations in an inhalation flow and/or an exhalation flow rate. Audible breathing incentive feedback may be provided in the form of a whistling sound, a vibratory sound, a clicking sound, or some combination thereof, in any of the manners described herein in conjunction with FIGS. 3, 4, and 5.

With reference to FIGS. 3, 4, and 5, in some embodiments, providing breathing incentive feedback comprises providing audible feedback via an integrated breathing incentive 135 in response to the inhalation through patient interface 110. Audible feedback may be provided by diverted flow (e.g., a diverted flow of admitted ambient air 141) moving through an audible signal generator (e.g., 135B, 135C, 135D, or the like). For example, responsive to inhalation, in various embodiments an audible signal is generated with an audible signal generator that is integrated with nebulizer 100. In some embodiments, the audible signal varies in response to variations in the inhalation flow rate. One or more audible signal generators may be integrated with nebulizer 100. In one embodiment, as illustrated in FIG. 3, integrated breathing incentive 135B generates, via fluid mechanical motion of diverted flow, a whistling sound that varies in response to variations in the inhalation flow rate. In one embodiment, as illustrated in FIG. 4, integrated breathing incentive 135C generates a vibratory sound in response to inhalation, and the vibratory sound varies in response to variations in the inhalation flow rate. In one embodiment, as illustrated in FIG. 5, integrated breathing incentive 135D generates a clicking sound in response to inhalation induced rotation of a wheel, and the clicking sound varies in response to variations in the inhalation flow rate.

With reference to FIG. 7B, at 730 of flow diagram 700, the method as described in procedures 710 and 720 further comprises providing breathing incentive feedback which describes a propriety of an exhalation flow rate. Breathing incentive feedback with respect to an inhalation flow rate may be utilized in conjunction with visible and/or audible exhalation breathing incentive feedback. For example, an integrated breathing incentive 135 with a float similar to that which is illustrated in FIG. 2 may move in response to variations in an exhalation flow rate. Additionally or alternatively, an integrated breathing incentive 135 which comprises a color change material as illustrated in FIGS. 6A and 6B may be used to provide visible feedback regarding exhalation flow rate. Likewise, a breathing incentive 135 which provides audible feedback, such as a whistling sound, vibratory sound, or clicking sound (see e.g., FIGS. 3, 4, and 5), may be used to provide exhalation breathing incentive feedback. It is appreciated that a combination of audible and visible exhalation breathing incentive feedback may be provided.

### CONCLUSION

Various embodiments have been described in various combinations. However, any two or more embodiments may be combined. For example, two or more breathing incentives can be included in a nebulizer 100 to provide two or more mechanisms of visible breathing incentive feedback, two or more mechanisms of audible breathing incentive feedback, or some combination of visible and audible breathing incentive feedback, in regard to the inhalation flow rate of a patient and/or the exhalation flow rate of a patient. Further, any embodiment may be used separately from any other embodiment. Features, structures, or characteristics of any embodiment may be combined in any suitable manner with one or more other features, structures, or characteristics. For example, integrated breathing incentive 135A may be used in combination with a second integrated breathing incentive such as breathing incentive 135E, where both provide visible breathing incentive feedback. Similarly integrated breathing incentives 135A and/or 135E may be used in combination with one or more integrated breathing incentive (e.g., 135B, 135C, 135D) which provide audible breathing incentive feedback. Additionally, one or some combination of visible and/or audible breathing incentives may be used to provide feedback incentive regarding an inhalation flow rate, while additional breathing incentives are used to provide feedback regarding an exhalation flow rate.

Examples of the subject matter are thus described. Although the subject matter has been described in a language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A nebulizer comprising:
a body;
a patient interface coupled with said body;
an air inlet coupled with said body and configured for admittance of ambient air into said body in response to inhalation through said patient interface;
an air outlet coupled with said body and configured for discharging exhaled breath; and
at least one breathing incentive integrated with said nebulizer and configured to provide feedback in response to said inhalation, wherein said feedback describes a propriety of a flow rate of said inhalation so as to assist a patient using said nebulizer in achieving a therapeutically effective flow rate for delivery of a medical aerosol.

2. The nebulizer of claim 1, wherein said at least one breathing incentive comprises:
a movable float disposed within a housing and configured to move across a variety of positions in response to variations in said inhalation flow rate.

3. The nebulizer of claim 2, wherein said at least one breathing incentive further comprises:
a color changing material integrated with said nebulizer and configured to generate a visible color change which varies in response to variations in said inhalation flow rate.

4. The nebulizer of claim 2, wherein said at least one breathing incentive further comprises:
an audible signal generator configured to generate an audible signal which varies in response to variations in said inhalation flow rate.

5. The nebulizer of claim 2, wherein said housing further comprises:
visible graduations indicative of a range of positions of said movable float that are associated with a therapeutically effective flow rate for delivery of said medical aerosol.

6. The nebulizer of claim 1, wherein said at least one breathing incentive further comprises:
a color changing sensor integrated with said nebulizer and configured to generate a visible color change which varies in response to variations in said inhalation flow rate.

7. The nebulizer of claim 6, wherein said at least one breathing incentive further comprises:
an audible signal generator configured to generate an audible signal which varies in response to variations in said inhalation flow rate.

8. The nebulizer of claim 1, wherein said at least one breathing incentive comprises:
an audible signal generator configured to generate a whistling sound via fluid mechanical motion, wherein said whistling sound varies in response to variations in said inhalation flow rate.

9. The nebulizer of claim 1, wherein said at least one breathing incentive comprises:
an audible signal generator configured to generate a generating a clicking sound in response to inhalation induced rotation of a wheel, wherein said clicking sound varies in response to variations in said inhalation flow rate.

10. The nebulizer of claim 1, wherein said at least one breathing incentive comprises:
an audible signal generator configured to generate a generating a vibratory sound in response to inhalation, wherein said vibratory sound varies in response to variations in said inhalation flow rate.

11. A method of administering medical aerosol comprising:
providing medical aerosol to a patient through a patient interface of a nebulizer in response to inhalation by said patient through said patient interface; and
providing breathing incentive feedback via at least one breathing incentive integrated with said nebulizer, wherein said breathing incentive feedback describes a propriety of a flow rate of said inhalation so as to assist said patient in achieving a therapeutically effective flow rate for delivery of said medical aerosol.

12. The method as recited in claim 11, wherein said providing breathing incentive feedback via at least one breathing incentive integrated with said nebulizer further comprises:
providing breathing incentive feedback which describes a propriety of an exhalation flow rate.

13. The method as recited in claim 11, wherein said providing breathing incentive feedback via at least one breathing incentive integrated with said nebulizer comprises:
responsive to said inhalation, moving a movable float within a graduated housing integrated with said nebulizer, said float movable to a variety of positions in said housing in response to variations in said inhalation flow rate.

14. The method as recited in claim 13, wherein said providing breathing incentive feedback via at least one breathing incentive integrated with said nebulizer further comprises:
generating a visible color change with a color changing sensor integrated with said nebulizer and configured to generate a visible color change which varies in response to variations in said inhalation flow rate.

15. The method as recited in claim 13, wherein said breathing incentive feedback via at least one breathing incentive integrated with said nebulizer further comprises:
responsive to said inhalation, generating an audible signal with an audible signal generator integrated with said nebulizer, wherein said audible signal varies in response to variations in said inhalation flow rate.

16. The method as recited in claim 11, wherein said providing breathing incentive feedback via at least one breathing incentive integrated with said nebulizer comprises:
generating a visible color change with a color changing sensor integrated with said nebulizer and configured to generate a visible color change which varies in response to variations in said inhalation flow rate.

17. The method as recited in claim 16, wherein said providing breathing incentive feedback via at least one breathing incentive integrated with said nebulizer further comprises:
responsive to said inhalation, generating an audible signal with an audible signal generator integrated with said nebulizer, wherein said audible signal varies in response to variations in said inhalation flow rate.

18. The method as recited in claim 11, wherein said providing breathing incentive feedback via at least one breathing incentive integrated with said nebulizer comprises:
generating a whistling sound with an audible signal generator configured to generate a whistling sound via fluid mechanical motion, wherein said whistling sound varies in response to variations in said inhalation flow rate.

19. The method as recited in claim 11, wherein said providing breathing incentive feedback via at least one breathing incentive integrated with said nebulizer comprises:
generating a clicking sound with an audible signal generator configured to generate a generating a clicking sound in response to inhalation induced rotation of a wheel, wherein said clicking sound varies in response to variations in said inhalation flow rate.

20. The method as recited in claim 11, wherein said providing breathing incentive feedback via at least one breathing incentive integrated with said nebulizer comprises:
generating a vibratory sound with an audible signal generator configured to generate a generating a vibratory sound in response to inhalation, wherein said vibratory sound varies in response to variations said inhalation flow rate.

21. A nebulizer comprising:
a body;
a patient interface configured for providing medical aerosol to a patient in response to inhalation through said patient interface by said patient; and
at least one means integrated with said nebulizer and for providing feedback in response to said inhalation, wherein said feedback describes a propriety of a flow rate of said inhalation so as to assist a patient using said nebulizer in achieving a therapeutically effective flow rate for delivery of said medical aerosol.
